(19) United States
(12) Patent Application Publication
(10) Pub. No.: US 2005/0220877 A1
(43) Pub. Date: Oct. 6, 2005

(54) Bilayer tablet comprising an antihistamine and a decongestant

(76) Inventors: Ashish A. Patel, North Brunswick, NJ (US); Mintong Guo, Plainsboro, NJ (US); Amol Singh Matharu, Bedminster, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/815,127

(22) Filed: Mar. 31, 2004

(51) Int. Cl. A61K 9/24; A61K 9/14

(52) U.S. Cl. 424/472

(57) ABSTRACT

A pharmaceutical composition in the form of a bilayer tablet comprising: (a) a first discrete portion made with Formulation (A) which comprises a sympathomimetic drug, or a pharmaceutically acceptable salt thereof, and a first carrier base material which provides a sustained-release of the sympathomimetic drug or the pharmaceutically acceptable salt thereof, said first carrier base material comprising a mixture of: (i) a filler; (ii) a cellulose binder selected from the group consisting of hydroxypropyl methylcellulose, hydroxypropyl cellulose, and mixtures thereof, wherein the hydroxypropyl cellulose has a molecular weight of at least 80,000; (iii) ethylcellulose; (iv) a wax; and (v) a lubricant; and (b) a second discrete portion made with Formulation (B) which comprises a piperidinoalkanol compound, or a pharmaceutically acceptable salt thereof, and a second carrier base material which provides an immediate-release of the piperidinoalkanol or the pharmaceutically acceptable salt thereof, said second carrier base comprising a mixture of: (i)′ a sugar; (ii)′ a disintegrant; and (iii)′ a lubricant. The bilayer tablets exhibit acceptable content uniformity under USP requirements, resist lamination and have acceptable physical strength during the self life.

### FIELD OF THE INVENTION

The present invention provides a bilayer tablet composition comprising an antihistamine and a decongestant.

### BACKGROUND OF THE INVENTION

Multiple layered tablets are described, for example, in the following patents:

U.S. Pat. No. 4,996,061 describes a pharmaceutical composition in the form of a multiple-compression tablet comprising a discrete zone made from a formulation which provides sustained-release of a therapeutically-effective decongestant amount of a sympathomimetic drug and a discrete zone made from a different formulation which provides immediate-release of a therapeutically-effective antihistaminic amount of a piperidinoalkanol and, optionally, a therapeutically-effective decongestant amount of a sympathomimetic drug.

U.S. Pat. No. 4,999,226 describes a multi-layered tablet containing an ibuprofen layer, a piperidinoalkanol antihistamine layer, and a layer or layers containing conventional pharmaceutical excipients which is interspersed between the ibuprofen and piperidinoalkanol layer and serves to physically separate them.

U.S. Pat. No. 6,039,974 describes a bilayer tablet containing:
(a) a first discrete zone containing a sympathomimetic drug and a first carrier base, wherein said first carrier base material provides a sustained-release of the sympathomimetic drug; and
(b) a second discrete zone containing a piperidinoalkanol drug and a second carrier base material, wherein said second carrier base material provides an immediate-release of the piperidinoalkanol drug.

### SUMMARY OF THE INVENTION

The present invention provides a pharmaceutical composition in the form of a bilayer tablet comprising:
(a) a first discrete portion made with Formulation (A) which comprises a sympathomimetic drug, or a pharmaceutically acceptable salt thereof, and a first carrier base material which provides a sustained-release of the sympathomimetic drug or the pharmaceutically acceptable salt thereof, said first carrier base material comprising a mixture of: (i) a filler; (ii) a cellulose binder selected from the group consisting of hydroxypropyl methylcellulose, hydroxypropyl cellulose, and mixtures thereof, wherein the hydroxypropyl cellulose has a molecular weight of at least 80,000; (iii) ethylcellulose; (iv) a wax; and (v) a lubricant; and
(b) a second discrete portion made with Formulation (B) which comprises a piperidinoalkanol compound, or a pharmaceutically acceptable salt thereof, and a second carrier base material which provides an immediate-release of the piperidinoalkanol or the pharmaceutically acceptable salt thereof, said second carrier base comprising a mixture of: (i)′ a sugar; (ii)′ a disintegrant; and (iii)′ a lubricant.

The bilayer tablets of the invention provide immediate absorption, and bioavailability of a piperidinoalkanol compound, such as fexofenadine, and efficient sustained-release and bioavailability of a sympathomimetic drug, such as pseudoephedrine hydrochloride, after oral administration thereof. In addition, the bilayer tablets exhibit acceptable content uniformity under USP requirements, resist lamination and have acceptable physical strength during the self life.

### DESCRIPTION OF THE INVENTION

The invention provides a pharmaceutical composition in the form of a bilayer tablet comprising:
(a) a first discrete portion made with Formulation (A) which comprises a sympathomimetic drug, or a pharmaceutically acceptable salt thereof, and a first carrier base material which provides a sustained-release of the sympathomimetic drug or the pharmaceutically acceptable salt thereof, said first carrier base material comprising a mixture of: (i) a filler; (ii) a cellulose binder selected from the group consisting of hydroxypropyl methylcellulose, hydroxypropyl cellulose, and mixtures thereof, wherein the hydroxypropyl cellulose has a molecular weight of at least 80,000; (iii) ethylcellulose; (iv) a wax; and (v) a lubricant; and
(b) a second discrete portion made with Formulation (B) which comprises a piperidinoalkanol compound, or a pharmaceutically acceptable salt thereof, and a second carrier base material which provides an immediate-release of the piperidinoalkanol or the pharmaceutically acceptable salt thereof, said second carrier base comprising a mixture of: (i)′ a sugar; (ii)′ a disintegrant; and (iii)′ a lubricant.

As used herein, “bilayer tablet” is a tablet which is made up of two or more distinct layers or discrete zones of granulation compressed together with the individual layers lying one on top of another. Bilayer tablets have the appearance of a sandwich because the edges of each layer or zone is exposed. Such bilayer tablets are generally prepared by compressing a granulation onto a previously compressed granulation. The operation may be repeated to produce bilayer tablets of more than two layers. In a preferred embodiment of the present invention, the bilayer tablet consists of two layers wherein one layer is made from Formulation (A) and the other layer is made from Formulation (B).

Sympathomimetic drugs include, but are not limited to, pseudoephedrine, phenylephrine and phenylpropanolamine. The sympathomimetic drugs can be used as free amines or as pharmaceutically acceptable salts thereof. Sympathomimetic drugs are useful in providing relief of nasal congestion. Preferably, the sympathomimetic drug is pseudoephedrine hydrochloride.

Piperidinoalkanol compounds are useful as antihistamines, anti-allergy agents and bronchodilators. The piperidinoalkanol compounds and their pharmaceutically acceptable salts refers to compounds having formulae (I), (II) and (III) as follows: 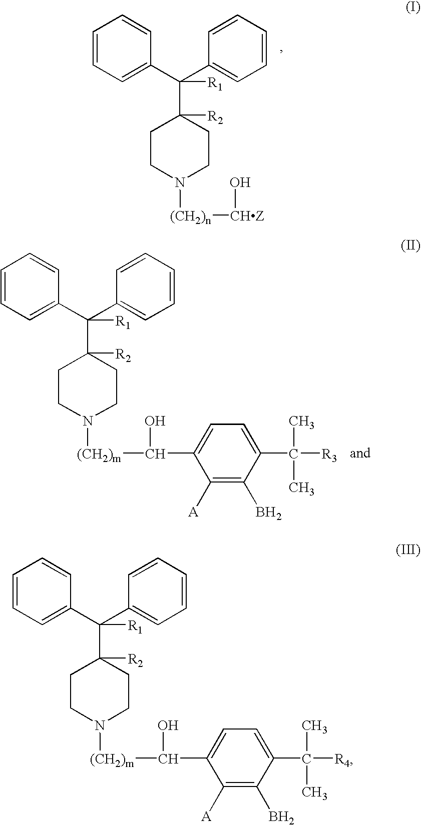
wherein
R₁ is hydrogen or hydroxy;
R₂ is hydrogen or
R₁ and R₂, taken together , form a second bond between the carbon atoms bearing R₁ and R₂;
R₃ is —CH₃, or —CH₂OH, each A and B is hydrogen or hydroxy, with the provisos that at least one of A or B is hydrogen and one of A or B is other than hydrogen when R₃ is —CH₃ and pharmaceutically acceptable salts and individual optical isomers thereof;
R₄ is —COOH or —COO alkyl, wherein the alkyl moiety has from 1-6 carbon atoms and is straight or branched, each of A and B is hydrogen or hydroxy, with the proviso that at least one of A or B is hydrogen; and pharmaceutically acceptable salts and individual optical isomers thereof;
Z is thienyl, phenyl or substituted phenyl wherein the substituents on the substituted phenyl may be attached at the ortho, meta or para positions of the unsubstituted phenyl ring and are selected from a halogen, straight or branched alkyl moiety having 1-4 carbon atoms, alkoxy moiety having 1-4 carbon atoms, dialkylamino group or a saturated monocyclic heterocyclic ring selected from the group consisting of pyrolidino, piperidino, morpholino or N-alkylpiperizino, or pharmaceutically acceptable acid addition salts thereof, wherein the alkyl moiety has 1-4 carbon atoms;
m is an integer of from 1-5; and
n is an integer of from 1-3.

A preferred piperidinoalkanol compound is 4-[4-[4-(hydroxydiphenylmethyl)-1-piperidinyl]-1-hydroxybutyl]-α,α-dimethylbenzeneacetic acid hydrochloride, also known as fexofenadine hydrochloride, of formula (IIIa) as follows: 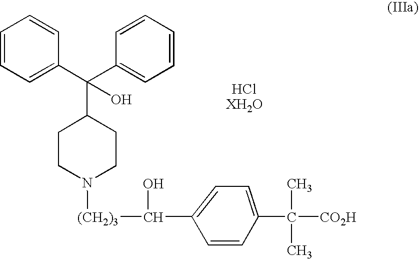
wherein X is a number ranging from about 0 to about 5, and the individual optical isomers thereof. Fexofenadine, wherein X is 0 or 1 in formula (IIIa) is the most preferred piperidinoalkanol compound.

In addition, a preferred piperidinoalkanol compound is the free base of 4-[4-[4-(hydroxydiphenylmethyl)-1-piperidinyl]-1-hydroxybutyl]-α,α-dimethylbenzeneacetic acid of formula (IIIb) as follows: 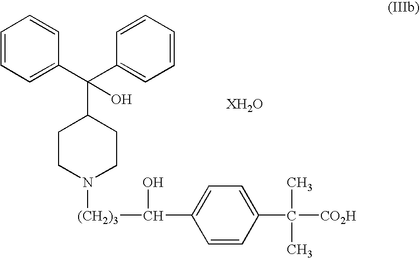
wherein X is a number ranging from about 0 to about 5, and the individual optical isomers thereof.

Included within the scope of the present invention are the pseudomorphs and polymorphs of the hydrated and anhydrous free base of 4-[4-[4-(hydroxydiphenylmethyl)-1-piperidinyl]-1-hydroxybutyl]-α,α-dimethylbenzeneacetic acid.

Pharmaceutically acceptable salts of piperidinoalkanol compounds refers to those salts of Formulae (I), (II), (III) and (IIIa) that are not substantially toxic at the dosage administered to achieve the desired effect and do not independently possess significant pharmacological activity. The salts included within the scope of this term are pharmaceutically acceptable acid addition salts of a suitable inorganic or organic acid. Suitable inorganic acids are, e.g., hydrochloric, hydrobromic, sulfuric and phosphoric acids. Suitable organic acids include carboxylic acids, such as acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acid; and sulfonic acids, such as methanesulfonic, ethanesulfonic and β-hydroxyethanesulfonic acid.

In addition, pharmaceutically acceptable salts of piperidinoalkanol compounds include those salts of Formulae (I), (II), (III) and (IIIa) formed with inorganic and organic bases, such as those of alkali metals, e.g., sodium, potassium and lithium; alkaline earth metals, e.g., calcium and magnesium; light metals of Group IIIA, e.g., aluminum; organic amines, e.g., primary, secondary or tertiary amines, such as cyclohexylamine, ethylamine, pyridine, methylaminoethanol and piperazine. The salts are prepared by conventional means known by one of ordinary skill in the art as, e.g., by treating a piperidinoalkanol compound of Formula (I), (II), (III) or (IIIa) with an appropriate acid or base. Such salts can exist in either a hydrated or substantially anhydrous form. The preferred acid addition salts are those prepared from hydrochloric acid, sulfuric acid and tartaric acid.

The first discrete portion of the bilayer tablet is made with Formulation (A) comprises a sympathomimetic drug, or a pharmaceutically acceptable salt thereof, and a first carrier base material. The first carrier base material comprises a mixture of: (i) a filler; (ii) a cellulose binder selected from the group consisting of hydroxypropyl methylcellulose, hydroxypropyl cellulose, and mixtures thereof, wherein the hydroxypropyl cellulose has a molecular weight of at least 80,000; (iii) ethylcellulose; (iv) a wax; and (v) a lubricant.

The filler in Formulation (A) is preferably selected from lactose; sucrose; dextrose; starch; pre-gelatinized starch; polyols, such as mannitol, sorbitol and xylitol; cellulose, such as microcrystalline cellulose; and inorganic salts, such as dibasic calcium phosphate, tribasic calcium phosphate and calcium sulfate. A mixture of fillers may also be used. Preferably, the filler is lactose monohydrate.

The amount of filler in Formulation (A) is preferably from about 1 weight percent (wt. %) to about 30 wt. %, more preferably, from about 5 wt. % to about 20 wt. %, based on the total weight of formulation (A). Most preferably, the amount of filler is from about 5 wt. % to about 10 wt. %, based on the total weight of Formulation (A).

The cellulose binder in Formulation (A) is selected from hydroxypropyl methylcellulose and hydroxypropyl cellulose having a molecular weight of at least 80,000. A mixture of cellulose binders may also be used. The amount of cellulose binder is preferably from about 10 wt. % to about 60 wt. %, more preferably, about 20 wt. % to about 50 wt. %, based on the total weight of Formulation (A). Most preferably, the amount of cellulose binder is from about 30 wt. % to about 40 wt. %, based on the total weight of Formulation (A).

The amount of ethylcellulose in Formulation (A) is preferably from about 5 wt. % to about 50 wt. %, more preferably, about 10 wt. % to about 35 wt. %, based on the total weight of Formulation (A). Most preferably, the amount of ethylcellulose is about 24 wt. %, based on the total weight of Formulation (A).

The wax in Formulation (A) is preferably selected from stearyl alcohol or cetyl alcohol, carnuba wax, white wax, yellow wax and microcrystalline wax. A mixture of waxes may also be used. More preferably, the wax is stearyl alcohol.

The amount of wax in Formulation (A) is preferably from about 2 wt. % to about 50 wt. %, more preferably, about 10 wt. % to about 30 wt. %, based on the total weight of Formulation (A). Most preferably, the amount of wax is about 24 wt. %, based on the total weight of Formulation (A).

The lubricant in Formulation (A) is preferably selected from vegetable oils, such as hydrogenated vegetable oil or hydrogenated castor oil; polyethylene glycols, such as PEG-4000 and PEG-6000; stearic acid; salts of stearic acid, such as calcium stearate, magnesium stearate, sodium stearate, and sodium stearyl fumarate. A mixture of lubricants may also be used. A preferred lubricant is magnesium stearate.

The amount of the lubricant in Formulation (A) is preferably from about 0.1 wt. % to about 3 wt. %, based on the total weight of Formulation (A). More preferably, the amount of the lubricant is from about 0.5 wt. % to about 2 wt. %, most preferably about 1.2 wt. %, based on the total weight of Formulation (A).

The second discrete portion of the bilayer tablet is made with Formulation (B) which comprises a piperidinoalkanol, or a pharmaceutically acceptable salt thereof, and a second carrier base material. The second carrier base material comprises a mixture of: (i)′ a sugar; (ii)′ a disintegrant; and (iii)′ a lubricant.

The sugar in Formulation (B) includes monosaccharides and disaccharides. The sugar is preferably selected from lactose, mannitol, sorbitol, sucrose, dextrose, maltose, and fructose. A mixture or combination of sugars may also be used. Preferably, the sugar is lactose.

The lactose that is preferred as a sugar in Formulation (B) is preferably selected from lactose monohydrate, lactose anhydrous, α-lactose and β-lactose. A mixture of lactose may also be used. Preferably, the lactose is lactose monohydrate.

The amount of sugar in Formulation (B) is preferably from about 10 wt. % to about 70 wt. %, more preferably, from about 25 wt. % to about 65 wt. %, based on the total weight of Formulation (B). Most preferably, the amount of sugar is from about 50 wt. % to about 60 wt. %, based on the total weight of Formulation (B).

The disintegrant in Formulation (B) is preferably selected from starch and starch derivatives, including cross-linked sodium salt of a carboxymethyl ether of starch, such as sodium starch glycolate; pre-gelatinized starch, such as Starch 1500; low substituted hydroxypropyl cellulose; cross-linked sodium carboxymethyl cellulose, such as croscarmellose sodium; cross-linked polyvinylpyrrolidone, such as crospovidone; and microcrystalline cellulose. A mixture of disintegrants may also be used. Preferably, the disintegrant is low substituted hydroxypropyl cellulose.

The low-substituted hydroxypropyl cellulose (L-HPC) that is preferred as a disintegrant in Formulation (B) is available in a number of different grades which have different particle sizes and substitution levels, and which are classified on the basis of their % hydroxypropoxy content. When dried at 105° C. for 1 hour, the L-HPC contains from about 5% to about 16% of hydroxypropoxy groups, preferably from about 10% to about 13% of hydroxypropoxy groups. Suitable grades of L-HPC include the following:
1) LH-11 having a hydroxypropoxy content of 11% and an average particle size of 50 microns;
2) LH-21 having a hydroxypropoxy content of 11% and an average particle size of 40 microns;
3) LH-31 having a hydroxypropoxy content of 11% and an average particle size of 25 microns;
4) LH-22 having a hydroxypropoxy content of 8% and an average particle size of 40 microns;
5) LH-32 having a hydroxypropoxy content of 8% and an average particle size of 25 microns;
6) LH-20 having a hydroxypropoxy content of 13%, and an average particle size of 40 microns; and
7) LH-30 having a hydroxypropoxy content of 13%, and an average particle size of 25 microns.

A preferred L-HPC is commercially-available from Shin-Etsu Chemical Company under the trade designation L-HPC Grade LH-21. A mixture of L-HPC's may also be used.

The amount of the disintegrant in Formulation (B) is preferably from about 1 wt. % to about 40 wt. %, based on the total weight of Formulation (B). More preferably, the amount of the disintegrant is from about 5 wt. % to about 25 wt. %, most preferably about 10 wt. % to about 15 wt. %, based on the total weight of Formulation (B).

The lubricant in Formulation (B) is preferably selected from vegetable oils, such as hydrogenated vegetable oil or hydrogenated castor oil; polyethylene glycols, such as PEG-4000 and PEG-6000; stearic acid; salts of stearic acid, such as calcium stearate, magnesium stearate, sodium stearate, and sodium stearyl fumarate. A mixture of lubricants may also be used. A preferred lubricant is magnesium stearate.

The amount of the lubricant in Formulation (B) is preferably from about 0.1 wt. % to about 3 wt. %, based on the total weight of Formulation (B). More preferably, the amount of the lubricant is from about 0.5 wt. % to about 2 wt. %, most preferably about 1 wt. %, based on the total weight of Formulation (B).

In a preferred embodiment of the invention, the first carrier base material in Formulation (A) comprises a mixture of: (i) lactose monohydrate; (ii) hydroxypropyl methylcellulose; (iii) ethylcellulose; (iv) stearyl alcohol; and (v) magnesium stearate.

In a preferred embodiment of the invention, the second carrier base material in Formulation (B) comprises a mixture of: (i)′ lactose; (ii)′ low-substituted hydroxypropyl cellulose; and (iii)′ magnesium stearate.

In a preferred embodiment, the bilayer tablet composition of the invention is essentially free of a glidant. As used herein, “essentially free” means that the bilayer tablet composition contains less than 3.5 wt. %, more preferably less than 1 wt. %, based on the weight of the composition of a glidant. Most preferably, the bilayer tablet composition does not contain a glidant. Examples of glidants include silica, silicon dioxide, colloidal silicon dioxide, fumed silicon dioxide, magnesium trisilicate, powdered cellulose, starch, talc and tribasic calcium phosphate.

It is understood that a therapeutically effective decongestant amount of a sympathomimetic drug is present in Formulation (A). The carrier base material of Formulation (A) provides a prolonged or sustained-release of the active medicament whereas the carrier base material of Formulation (B) provides an immediate-release of the active medicament. As used herein the term “sustained-release” refers to a property of the pharmaceutical composition wherein the absorption and bioavailability of the active medicament is maintained in a time-release pattern such that therapeutically effective decongestant amounts of the sympathomimetic drug are bioavailable over an extended period of time. As used herein the term “immediate-release” refers to a property of the pharmaceutical composition wherein the entire dose of active medicament is made bioavailable without substantial delay.

A therapeutically-effective decongestant amount of a sympathomimetic drug is that amount which produces the desired decongestant therapeutic response upon oral administration and can be readily determined by one skilled in the art by use of conventional techniques and by observing results obtained under analogous circumstances. In determining the therapeutically effective decongestant amount or dose, a number of factors are considered by the attending diagnostician including, but not limited to, the species of mammal, its size, age and general health, the response of the individual patient, the particular compound administered, the mode of administration, the bioavailability characteristics of the preparation administered, the dose regimen selected, the use of concomitant medication and other relevant circumstances.

A therapeutically-effective decongestant amount of a sympathomimetic drug will vary from about 5 mg to about 240 mg. Preferred amounts will vary from about 60 mg to about 150 mg, with about 120 mg administered twice daily being most preferred.

A therapeutically-effective anti-histaminic amount of a piperidinoalkanol compound of formulae (I)-(IIIb) is that amount which produces the desired therapeutic response, i.e., anti-histaminic, anti-allergic, bronchodilatory effect or reduction or elimination of urticaria, upon oral administration according to a single- or multiple-dosage regimen. A therapeutically-effective anti-histaminic amount of a piperidinoalkanol compound of formulae (I)-(IIIb) may vary over a wide range is from about 5 mg to about 240 mg. The preferred therapeutically-effective anti-histaminic amount of a piperidinoalkanol compound of formulae (I)-(IIIb) will vary from about 20 mg to about 70 mg with about 60 mg administered twice daily being most preferred.

In a preferred embodiment of the invention, with respect to the piperidinoalkanol in Formulation (B), about 60 mg of fexofenadine hydrochloride is preferred. In a preferred embodiment of the present invention, with respect to the sympathomimetic drug in Formulation (A), about 120 mg of pseudoephedrine hydrochloride is preferred.

Formulations (A) and (B), of the pharmaceutical compositions of the present invention, optionally may contain one or more additional pharmaceutically acceptable excipients. Examples of such additional excipients are surfactants, coating agents, diluents, anti-caking agents, amino acids, fibers, solubilizers, disintegrants, fillers, lubricants, emulsifiers, buffers, stabilizers, dyes, anti-oxidants, anti-adherents, preservatives, electrolytes and carrier materials. A combination of additional excipients may also be used. Such additional excipients are known to those skilled in the art, and thus, only a limited number will be specifically referenced.

Examples of binders include, but are not limited to, cellulose derivatives, such as microcrystalline cellulose, methylcellulose, carboxymethycellulose sodium, hydroxypropyl methylcellulose, hydroxyethyl cellulose and hydroxypropyl cellulose; polyvidone; polyvinyl pyrrolidone; gelatin; natural gums, such as acacia, tragacanth, guar and pectin; starch paste; pre-gelatinized starch; sucrose; corn syrup; polyethylene glycols and sodium alginate; ammonium calcium alginate; magnesium aluminum silicate; and polyethylene glycols.

Several co-processed filler-binders are commercially-available, including cellactose (α-lactose monohydrate and powdered cellulose 75:25), microcelac (α-lactose monohydrate and powdered cellulose 75:25), ludipress (93% α-lactose monohydrate, 3.5% polyvinylpyrrolidone and 3.5% crospovidone) and pharmatose DCL 40 (95% β-lactose and 5% lactitol).

The bilayer tablets of the invention are especially useful as antihistamines, anti-allergy agents, bronchodilators and in the treatment of urticaria.

The following non-limiting examples illustrate further aspects of the invention.

### EXAMPLE 1

Preparation of a fexofenadine/pseudoephedrine
bilayer tablet composition.

| Ingredient | %/Layer | (mg/Tablet) |
|---|---|---|
| Formulation (A): | | |
| Pseudoephedrine HCl | 24.0 | 120.0 |
| Lactose Monohydrate | 7.3 | 36.5 |
| Hydroxypropyl | 36.0 | 180.0 |
| Methylcellulose Type 2208 | | |
| Stearyl Alcohol | 24.0 | 120.0 |
| Ethylcellulose | 7.50 | 37.5 |
| Magnesium Stearate | 1.2 | 6.0 |
| Formulation (A) Weight | 100% | 500 mg |
| Formulation (B): | | |
| Fexofenadine HCl | 30.0 | 60.0 |
| Lactose Monohydrate | 57.5 | 115.0 |
| HPC LH-21 | 11.5 | 7.0 |
| Magnesium Stearate | 1.0 | 2.0 |
| Formulation (B) Weight | 100% | 200 mg |
| Coated Tablets: | | |
| Opadry ® Clear YS-1-7006 | 2.0 | 14.0 |
| Purified Water | None | q.s. |
| Coated Tablet Weight | 100% | 714 mg |

A first discrete portion made with Formulation (A) was prepared as follows:

A pre-mix was prepared which contained pseudoephedrine HCl, lactose monohydrate and hydroxypropyl methylcellulose, using a planetary mixer at 50 rpm for 15 minutes. Separately, stearyl alcohol was melted in a planetary mixer with jacketed bowl and Cromalax Temperature Control System set at 80° C. and 50 rpm. Ethylcellulose was combined with the melted stearyl alcohol in the planetary mixer with jacketed bowl and Cromalax Temperature Control System set at 80° C. and 50 rpm, and mixed until a clear paste was formed. The pre-mix formed above was combined with the stearyl alcohol and ethylcellulose paste in the planetary mixer with jacketed bowl and Cromalax Temperature Control System set at 80° C. and 50 rpm. The resulting melt granulation was cooled to less than 30° C. using a tray dryer with drying trays. The dried granulation was milled using a Fitz-Mill equipped with a #65 screen at medium speed. Magnesium stearate was added to the dried granulation through hand screen #20 and mixed using a BOHLE Blender for 10 minutes to form a final mix.

A second discrete portion made with Formulation (B) was prepared as follows:

A pre-mix was prepared using a 800 L Fielder mixer having a plough speed setting #1, chopper speed setting #1 for 5 minutes, which contained fexofenadine HCl, lactose and low substituted hydroxypropyl cellulose. Purified water was added to the pre-mix to form a wet granulation. The wet granulation was dried using a tray dryer with drying trays at 130° F. The dried granulation was milled using a Quadro Co-Mill equipped with a #75 screen. Low substituted hydroxypropyl cellulose was added to the milled granulation and mixed using a 566 L Patterson-Kelley Twinshell Blender for 15 minutes. Magnesium stearate was added through hand screen #20 and mixed using the Twinshell Blender for 3 minutes to form a final mix.

The bilayer tablet was formed using Korsch XL 400 Tablet Press equipped with bilayer kit. The first discrete portion containing Formulation (A) was compressed at a target layer weight of 500 mg. The second discrete portion containing Formulation (B) was compressed on top of the first discrete portion at a target layer weight of 200 mg. The resulting bilayer tablet weighed 700 mg.

The compressed bilayer tablets were coated for appearance in a 48 inch Accela-Cota using a coating solution of Opadry® Clear YS-1-7006. Each coated bilayer tablet weight approximately 714 mg. Thus, the coating provided about 2% weight gain, based on the total tablet weight.

While the invention has been described with particular reference to certain embodiments thereof, it will be understood that changes and modifications may be made by those of ordinary skill within the scope and spirit of the following claims:

1. A pharmaceutical composition in the form of a bilayer tablet comprising:
(a) a first discrete portion made with Formulation (A) which comprises a sympathomimetic drug, or a pharmaceutically acceptable salt thereof, and a first carrier base material which provides a sustained-release of the sympathomimetic drug or the pharmaceutically acceptable salt thereof, said first carrier base material comprising a mixture of: (i) a filler; (ii) a cellulose binder selected from the group consisting of hydroxypropyl methylcellulose, hydroxypropyl cellulose, and mixtures thereof, wherein the hydroxypropyl cellulose has a molecular weight of at least 80,000; (iii) ethylcellulose; (iv) a wax; and (v) a lubricant; and
(b) a second discrete portion made with Formulation (B) which comprises a piperidinoalkanol compound, or a pharmaceutically acceptable salt thereof, and a second carrier base material which provides an immediate-release of the piperidinoalkanol or the pharmaceutically acceptable salt thereof, said second carrier base comprising a mixture of: (i)′ a sugar; (ii)′ a disintegrant; and (iii)′ a lubricant.

2. The composition according to claim 1, wherein the sympathomimetic drug is selected from the group consisting of pseudoephedrine, phenylephrine, phenylpropanolamine and pharmaceutically acceptable salts thereof.

3. The composition according to claim 2, wherein the sympathomimetic drug is pseudoephedrine hydrochloride.

4. The pharmaceutical composition according to claim 1, wherein the piperidinoalkanol compound has a formula selected from the group consisting of Formulae (I), (II) and (III), as follows: 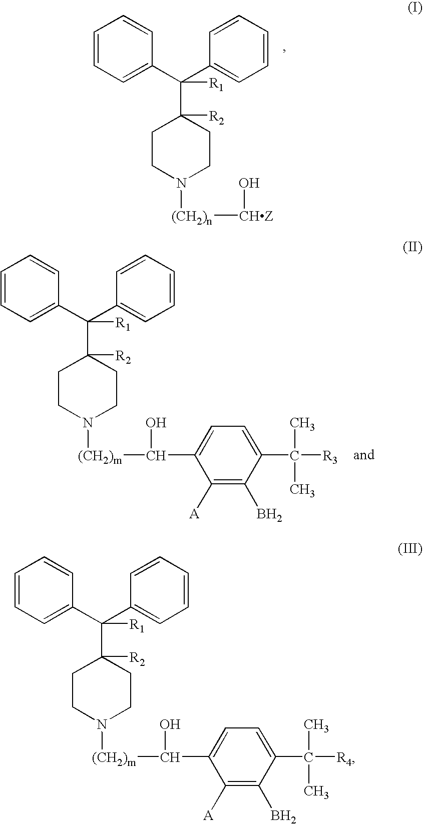
wherein
R₁ is hydrogen or hydroxy;
R₂ is hydrogen or
R₁ and R₂, taken together, form a second bond between the carbon atoms bearing R₁ and R₂;
R₃ is —CH₃, or —CH₂OH, each A and B is hydrogen or hydroxy, with the provisos that at least one of A or B is hydrogen and one of A or B is other than hydrogen when R₃ is —CH₃ and pharmaceutically acceptable salts and individual optical isomers thereof;
R₄ is —COOH or —COO alkyl, wherein the alkyl moiety has from 1-6 carbon atoms and is straight or branched, each of A and B is hydrogen or hydroxy, with the proviso that at least one of A or B is hydrogen; and pharmaceutically acceptable salts and individual optical isomers thereof;
Z is thienyl, phenyl or substituted phenyl wherein the substituents on the substituted phenyl may be attached at the ortho, meta or para positions of the unsubstituted phenyl ring and are selected from the group consisting of a halogen, a straight or branched alkyl moiety having 1-4 carbon atoms, an alkoxy moiety having 1-4 carbon atoms, a dialkylamino group or a saturated monocyclic heterocyclic ring selected from the group consisting of pyrolidino, piperidino, morpholino or N-alkylpiperizino, or pharmaceutically acceptable acid addition salts thereof, wherein the alkyl moiety has 1-4 carbon atoms;
m is an integer from 1 to 5; and
n is an integer from 1 to 3.

5. The composition according to claim 4, wherein the piperidinoalkanol compound is 4-[4-[4-(hydroxydiphenylmethyl)-1-piperidinyl]-1-hydroxybutyl]-α,α-dimethylbenzeneacetic acid hydrochloride.

6. The composition according to claim 5, wherein the piperidinoalkanol compound has Formula (IIIa) as follows: 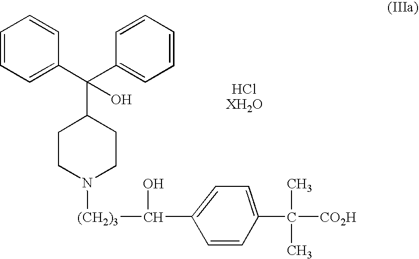
wherein X is a number from about 0 to about 5.

7. The composition according to claim 5, wherein the piperidinoalkanol compound has Formula (IIIb) as follows: 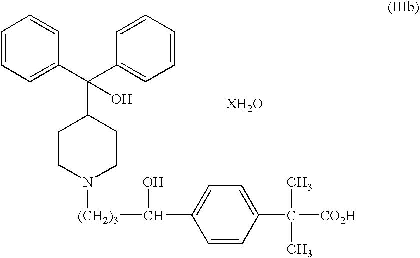
wherein X is a number from about 0 to about 5.

8. The composition according to claim 1, wherein the filler in Formulation (A) is selected from the group consisting of lactose, sucrose, dextrose, starch, pre-gelatinized starch, mannitol, sorbitol, xylitol, microcrystalline cellulose, dibasic calcium phosphate, tribasic calcium phosphate, calcium sulphate and mixtures thereof.

9. The composition according to claim 1, wherein the wax in Formulation (A) is selected from the group consisting of stearyl alcohol, cetyl alcohol, carnuba wax, white wax, yellow wax, microcrystalline wax, and mixtures thereof.

10. The composition according to claim 1, wherein the lubricant in Formulation (A) is selected from the group consisting of hydrogenated vegetable oil, hydrogenated castor oil, polyethylene glycol, stearic acid, calcium stearate, magnesium stearate, sodium stearate, sodium stearyl fumarate, and mixtures thereof.

11. The composition according to claim 1, wherein the first carrier base material in Formulation (A) comprises a mixture of: (i) lactose monohydrate; (ii) hydroxypropyl methylcellulose; (iii) ethylcellulose; (iv) stearyl alcohol; and (v) magnesium stearate.

12. The composition according to claim 1, wherein the sugar in Formulation (B) is selected from the group consisting of lactose, mannitol, sorbitol, sucrose, dextrose, maltose, fructose, and mixtures thereof.

13. The composition according to claim 1, wherein the disintegrant in Formulation (B) is selected from the group consisting of starch, sodium starch glycolate, pre-gelatinized starch, low substituted hydroxypropyl cellulose, croscarmellose sodium, cross-linked polyvinylpyrrolidone, microcrystalline cellulose, and mixtures thereof.

14. The composition according to claim 1, wherein the disintegrant is a low-substituted hydroxypropyl cellulose.

15. The composition according to claim 14, wherein the low-substituted hydroxypropyl cellulose is selected from the group consisting of: LH-11 having a hydroxypropoxy content of 11% and an average particle size of 50 microns; LH-21 having a hydroxypropoxy content of 11% and an average particle. size of 40 microns; LH-31 having a hydroxypropoxy content of 11%, and an average particle size of 25 microns; LH-22 having a hydroxypropoxy content of 8%, and an average particle size of 40 microns; LH-32 having a hydroxypropoxy content of 8%, and an average particle size of 25 microns; LH-20 having a hydroxypropoxy content of 13%, and an average particle size of 40 microns; and LH-30 having a hydroxypropoxy content of 13%, and an average particle size of 25 microns.

16. The composition according to claim 1, wherein the lubricant in Formulation (B) is selected from the group consisting of hydrogenated vegetable oil, hydrogenated castor oil, polyethylene glycol, stearic acid, calcium stearate, magnesium stearate, sodium stearate, sodium stearyl fumarate, and mixtures thereof.

17. The composition according to claim 1, wherein the second carrier base material in Formulation (B) comprises a mixture of: (i)′ lactose; (ii)′ low-substituted hydroxypropyl cellulose; and (iii)′ magnesium stearate.

18. A pharmaceutical composition in the form of a bilayer tablet comprising:
(a) a first discrete portion made with Formulation (A) which comprises a sympathomimetic drug, or a pharmaceutically acceptable salt thereof, and a first carrier base material which provides a sustained-release of the sympathomimetic drug or the pharmaceutically acceptable salt thereof, said first carrier base material comprising a mixture of: (i) from about 1 wt. % to about 30 wt. % of a filler; (ii) from about 10 wt. % to about 60 wt. % of a cellulose binder selected from the group consisting of hydroxypropyl methylcellulose, hydroxypropyl cellulose, and mixtures thereof, wherein the hydroxypropyl cellulose has a molecular weight of at least 80,000; (iii) from about 5 wt. % to about 50 wt. % of ethylcellulose; (iv) from about 2 wt. % to about 50 wt. % of a wax; and (v) from about 0.1 wt. % to about 3 wt. % of a lubricant, wherein the weight percents in the first discrete portion are based on the total weight of Formulation (A); and
(b) a second discrete portion made with Formulation (B) which comprises a piperidinoalkanol compound, or a pharmaceutically acceptable salt thereof, and a second carrier base material which provides an immediate-release of the piperidinoalkanol or the pharmaceutically acceptable salt thereof, said second carrier base comprising a mixture of: (i)′ from about 10 wt. % to about 70 wt. % of a sugar;
(ii)′ from about 1 wt. % to about 40 wt. % of a disintegrant; and (iii)′ from about 0.1 wt. % to about 3 wt. % of a lubricant, wherein the weight percents in the second discrete portion are based on the total weight of Formulation (B).

19. The composition according to claim 18, wherein the filler in Formulation (A) is present in an amount from about 5 wt. % to about 20 wt. %.

20. The composition according to claim 1, wherein the cellulose binder in Formulation (A) is present in an amount from about 20 wt. % to about 50 wt. %.

21. The composition according to claim 1, wherein the ethylcellulose in Formulation (A) is present in an amount from about 10 wt. % to about 35 wt. %.

22. The composition according to claim 1, wherein the wax in Formulation (A) is present in an amount from about 10 wt. % to about 30 wt. %.

23. The composition according to claim 1, wherein the lubricant in Formulation (A) is present in an amount from about 0.5 wt. % to about 2 wt. %.

24. The composition according to claim 1, wherein the sugar in Formulation (B) is present in an amount from about 25 wt. % to about 65 wt. %.

25. The composition according to claim 1, wherein the disintegrant in Formulation (B) is present in an amount of from about 5 wt. % to about 25 wt. %.

26. The composition according to claim 1, wherein the lubricant in Formulation (B) is present in an amount from about 0.5 wt. % to about 2 wt. %.

27. The composition according to claim 1 which is essentially free of a glidant.

28. The composition according to claim 1, wherein the bilayer tablet is coated.

29. The composition according to claim 1, wherein the piperidinoalkanol compound is fexofenadine hydrochloride which is present in an amount of about 60 mg and the sympathomimetic drug is pseudoephedrine hydrochloride which is present in an amount of about 120 mg.
